(19) United States
(12) Patent Application Publication
(10) Pub. No.: US 2026/0060625 A1
(43) Pub. Date: Mar. 5, 2026

(54) MAMMOGRAPHY APPARATUS AND METHOD FOR OPERATING A MAMMOGRAPHY APPARATUS

(71) Applicant: Siemens Healthineers AG, Forchheim (DE)

(72) Inventors: Sebastian NEWRZELLA, Bayreuth (DE); Mohd MAHMEEN, Bangalore (IN); Jutta SPEITEL, Erlangen (DE)

(73) Assignee: Siemens Healthineers AG, Forchheim (DE)

(21) Appl. No.: 19/318,857

(22) Filed: Sep. 4, 2025

(30) Foreign Application Priority Data
Sep. 5, 2024 (DE) 10 2024 208 423.9

(51) Int. Cl. A61B 6/46; A61B 6/00; A61B 6/04; A61B 6/50; A61B 90/00

(52) U.S. Cl. CPC A61B 6/467; A61B 6/0414; A61B 6/502; A61B 6/54; A61B 90/08; A61B 2090/0807

(57) ABSTRACT

A mammography apparatus, comprises: a control device; an object support for placement of a breast of a patient; a compression device to compress the breast arranged on the object support; at least one operating element to at least one of increase or decrease a compression force acting upon the breast; and an acquisition device to acquire an item of compression information describing the compression force currently acting on the breast. The at least one operating element includes an actuator to output haptic information. The control device is configured to activate the actuator based on the item of compression information to generate haptic feedback.

### CROSS-REFERENCE TO RELATED APPLICATION(S)

The present application claims priority under 35 U.S.C. § 119 to German Patent Application No. 10 2024 208 423.9, filed Sep. 5, 2024, the entire contents of which are incorporated herein by reference.

### FIELD

One or more example embodiments of the present invention relate to a mammography apparatus comprising a control device, an object support for placement of a breast of an examination person, a compression device for compressing the breast arranged on the object support, at least one operating element for increasing and/or reducing a compression force acting upon the breast of the examination person and an acquisition device for acquiring a item of compression information, which defines the currently acting compression force. In addition, one or more example embodiments of the present invention relate to a method for operating a compression device.

### BACKGROUND

During a mammographic examination, the compression is a precondition both for the mammographic screening itself and also for the extraction of a biopsy. In particular, an adequate compression is a precondition for a sufficiently high image quality, since movements are prevented, a homogeneous breast thickness if achieved and a superposition of lesions is prevented. On the other hand, the mammographic examination must be tolerable to the patient. During the compression of the breast, a medical operating person, in particular, a medical technician must actuate an operating element in order to set the compression force that is exerted by a compression facility. The operating element can comprise, for example, a foot pedal, a twist knob, a lever or suchlike. The degree of the required compression depends upon the patient-specific application case. The operating person must therefore take account of different circumstances such as the compression force required for a best possible imaging result and the pain that the examination person can tolerate during the compression, in order to achieve the required compression force. This results in the operating person needing to monitor the examination person continuously, in addition to setting the compression force, in order to be sure that the pain is tolerable for the examination person. He must also keep track of the compression force shown on the display. This visual multitasking can result in a suboptimal concentration and an increased burden on the operating person.

According to the current state of the art, at least partially automatic and manually-assisted methods are known for finding a suitable compression, for example, under the name OpComp, as described in the white paper by Johannes Georg Korporaal entitled “Breast Compression with SoftSpeed and OpComp”, 2017 (retrievable under https://marketing.webassets.siemens-healthineers.com/180000000414065 2/Ocfc161cd72f/mammography-breast-compression-whitepaper_1800000004140652.pdf). In procedures of this type, the increase in the compression by the compression facility stops when a sufficient compression force has been achieved and the operating person must actuate the operating element again in order to continue with the compression. This leads to an indirect feedback loop in which the operating person does not feel the compression force directly himself, but rather must rely on feedback from the patient regarding her pain tolerance. At the same time, the display must be observed.

### SUMMARY

It is therefore an object of one or more example embodiments of the present invention to provide a mammography apparatus that is improved and more intuitive with regard to its operation.

In order to achieve at least this object, a mammography apparatus, according to one or more example embodiments of the present invention, of the type mentioned in the introduction is provided wherein the operating element has an actuator for the output of haptic information and the control facility (also referred to herein as a control device) is configured for activating the actuator dependent upon the item of compression information to generate a haptic feedback.

During the mammographic examination, the control facility analyzes the data acquired by the acquisition facility (also referred to herein as an acquisition device) relating to the item of compression information acquired by the acquisition facility, in real time. Control signals are generated by the control facility via hardware and/or software that is suitable for this, in particular algorithms, dependent upon the item of compression information and these can be output again for control of the actuator. The actuator then generates haptic properties and/or signals that serve the operating person as haptic feedback. This has the advantage that, dependent upon the increase and/or reduction of the compression force acting upon the breast of the examination person, a non-visual impression of the acting compression force is imparted to the operating person. It is thus easier for the operating person to estimate how great the compression force is and whether the compression force is still within a range that is tolerable to the examination person. In particular, it is no longer necessary to consult a display for indicating the compression force, which naturally can additionally be provided if needed, but rather the operating person can devote his entire attention to the examination person and to his reaction to the currently acting compression force.

In a development of one or more example embodiments of the present invention, the haptic feedback can comprise a vibration and/or a pulse output and/or a change in an actuation resistance of the operating element, wherein the actuator has a setting element for setting the actuation resistance and/or a motor arrangement for providing the vibration and/or the pulse. Depending upon the nature of the haptic feedback, different information items can be provided, as described below. For example, an actuating cylinder, in particular a spring-biased actuating cylinder, a pneumatic operating element and/or an electric motor can serve as the setting element for setting the actuation resistance.

Suitably, the control facility can be configured, for establishing the actuation resistance output via the actuator dependent upon the compression force, such that the actuation resistance increases as the compression force is increased and reduces as the compression force is reduced. Since the haptic feedback, in particular, the actuation resistance is intended to give to the operating person an intuitively understandable impression of how strong the compression force acting upon the breast is and therefore in which compression range the compression facility (also referred to as a compression device) is situated, it is suitable that the actuation resistance is accordingly increased on an increase of the compression force and reduced on a reduction of the compression force. The typical compression range of the breast in the context of a mammographic screening is usually between 50 N and 200 N, although dependent upon the country where it is used, the maximum compression force is between 90 N and 150 N. Accordingly, the actuation resistance can be adjusted by the control facility dependent upon the compression force such that, possibly with a certain offset, it lies in a particular ratio to the compression force applied. For example, the actuation resistance can correspond to 1.5 to 20 times the compression force, so that, in particular, an actuation resistance range from 1 N to 400 N is the result. If the actuation resistance corresponds, for example, to twice the compression force, on an increase in the compression force by 10 N, which corresponds to approximately 1 kg, there is an increase in the actuation resistance by at least 20 N, which corresponds to approximately 2 kg. If the compression force is reduced, the procedure can take place in a similar manner. This device that if the compression force is reduced by, for example, 10 N, with respect to the above example, the actuation resistance would also by reduced by 20 N. Thus, the operating person can effectively “feel” what compression force is currently being applied.

Preferably, the control facility can be configured for activating the actuator for vibration and/or pulse output when a notification condition is fulfilled that indicates that the compression force enters a compression range that is specific to a current imaging process. The compression range can be predetermined, although preferably at least the notification condition which determines the entry into the compression range is predefined. If the compression force exerted by the compression facility, through operation by the operating person, enters a compression range that is preferably specific to an image recording, in particular, a range in which the image quality for the detection of any mammary carcinomas or other lesions is correspondingly high, this can be notified to the operating person via a haptic feedback in the form of a vibration and/or a pulse output via the operating element. Accordingly, it can also be possible, on leaving the specific compression range, for a vibration and/or a pulse to be output again via the operating element, in particular, differentiable from the entry into the compression range. For example, on entry into the specific compression range, a vibration can be output and on leaving the compression range, a pulse can be output. This offers the advantage that the entry into and/or exit from the specific compression range cannot be confused since on entry into and/or exit from the compression range, different haptic signals are output. The notification of entry into the, in particular specific, compression range inspires the operating person, for example, to increased caution since the relevant range has now been reached. In addition or alternatively, it can also be provided that while the item of compression information indicates a compression force existing within the compression range, in particular, a soft vibration is continuously output. In this way, the operating person remains conscious that he has reached the, in particular, ideal compression range and, particularly advantageously, a differentiation between inside and outside is enabled.

The establishment of the compression range and/or of the notification condition describing the compression range in the predefined manner can take place in a variety of ways. The specific compression range can be different dependent upon the examination person, i.e. it can be different in different examination persons. It can also be dependent upon specific examination parameters, for example, it can be selected to be different for two-dimensional and three-dimensional measurements since with three-dimensional imaging, a lower compression of the breast can be sufficient for a diagnostic image quality. For example, different notification conditions can be specified for different dimensionalities.

Via a facility or device for acquiring parameters such as the change in breast thickness, that is, the deformation of the breast tissue, the achievement of a compression range specific to the examination object can be established, that is, the fulfillment of the notification condition. For example, the movement path of a movable compression plate of the compression facility can be fed back by way of a corresponding movement actuator system. The current compression force can also be established, in general, for example, via a corresponding compression force sensor, but also by other devices, mechanisms and/or means. For example, an approach can be selected that is comparable with the aforementioned previously mentioned OpComp technology. Suitably, the change in the breast thickness with the compression force is monitored, this describing the elastic deformation of the breast tissue. This deformation is specific to the examination person and is dependent, among other things, upon the thickness and the microstructure of the breast tissue. At the start, the degree of breast thickness change, that is, the effect of the increasing compression on the breast, is high but over time, that is, with increasing compression force, further reduces again. Beyond a certain point, a further compression would lead to only a very slight reduction in the breast thickness and thus only to a very slight enhancement of the image quality, but simultaneously to a severe increase in pain for the examination person. By way of a limit value for a change in the breast thickness, a compression force for the examination person can be established at which or from which, empirically, a good compromise is achieved between the image quality and the possible pain to the examination person. The notification condition therefore monitors whether a change in the breast thickness with an increase in the compression force, in particular, a corresponding gradient, falls below a predefined limit value.

According to a preferred development of one or more example embodiments of the present invention, the control facility can be configured, on fulfillment of the notification condition for changing from a first operating mode of the operating element, in particular, for continuous setting of the compression force, into a second operating mode, in particular, a stepped operating mode, provided the compression force remains in the compression range. With the notification regarding achievement of the, in particular, optimum compression range, in this way, the operating person is therefore also notified that an operating mode change is taking place. Preferably, the first operating mode can be provided for coarse setting and the second operating mode for fine setting. While the coarse setting can be made, in particular, steplessly, the fine setting is preferably made in steps by the operating person. The operating person has therefore reached a compression range that is assumed to be suitable for the examination but within which, in particular, by way of further compression, a further adaptation is suitable to reach an improved image quality. This further compression is at the discretion of the operating person. Accordingly, it is advantageous if, after reaching the specific compression range, an operating mode change can take place, following which, in a second operating mode specifically adjusted thereto, in particular, the stepped operating mode for fine setting, the operating person himself can adjust the further compression force. The setting of the compression force in the first operating mode can take place at least partially automatically, while in the second operating mode, the compression force can be set by the operating person and takes place, in particular, in predefined steps. This provides the advantage of a more precise and simultaneously gentler setting of the compression force, so that a best possible compromise between image quality and the wellbeing of the examination person can be achieved. Therein, both in the first and also the second operating mode, particularly advantageously, further haptic feedback can be output which intuitively conveys haptically the compression force and/or its change. In order to emphasize the change of the operating mode more emphatically, it can be provided that only after a waiting time, for example, 1 to 10 s, during which the operating element receives no input, it is ascertained, in particular, that the second operating mode is to be activated. It is further conceivable, on leaving the, in particular, specific compression range, also to deactivate the second operating mode again and, in particular, again after a waiting time, to return to the first operating mode.

Suitably, the control facility can be configured for increasing and/or reducing the compression force in steps on actuation of the operating element in at least one stepped operating mode of the operating element. As previously mentioned, the stepped operating mode can be provided, in particular, for the fine setting. It is conceivable that the step size can be set by the user. Thus, for example, via a specifying facility (also referred to as a specifying or input device), in particular, a touch display arranged on the mammography apparatus, a step size, in particular, in a range from 0.1 N to 2 N can be set by the operating person. It can be possible therein, even before the mammographic examination and/or at the start of the fine setting, to define the step size, but a change of the step size during the fine setting is also conceivable. This offers the advantage of a more precise and patient-specific fine setting of the compression force.

In a first variant, the actuation resistance that can be output by the actuator on non-actuation of the operating element can have a base value at least in the stepped operating mode, wherein the control facility is configured such that it increases the actuation resistance on operation of the operating element for increasing the compression force by the next step to an intermediate value and, on reaching the next step of the compression force, reduces the actuation resistance, in particular, at least substantially abruptly to the base value. If it is necessary, for example, in the context of the fine setting, to increase the compression force by one step, for example, by 2 N, in this first variant, the operating element can have a base value for the actuation resistance, on non-actuation, in particular, corresponding to between 0 N and 10 N, which corresponds to between 0 and 1 kg. On actuation of the operating element, in order to increase the compression force, the control facility can increase the actuation resistance by activating the actuator starting from the base value such that the operating person must, so to speak, overcome a threshold in order to reach the next compression step, that is, to increase the compression force by a value that corresponds to the step height. The intermediate value of the actuation resistance then effectively relates to the height of this threshold. It can be selected dependent upon the step height in order to represent it by way of the threshold. If the intermediate value is, for example, 10 N (corresponding to 1 kg), then the control facility can increase the actuation resistance up to a next compression step, preferably continuously, for example, linearly, with an actuation travel and/or an actuation time and/or step-wise, for example, in 1 N steps until the actuation resistance of 10 N as the intermediate value is reached, which preferably also corresponds to the moment at which the compression facility is activated to increase the compression force to the next compression step. Subsequently, the actuation resistance can fall, in particular, abruptly or at least with a steep flank until the actuation resistance again corresponds to the base value in order thus to notify the operating person that the increase operating command has been received and accepted. On increasing by the next compression step, the procedure repeats. If actuation times are used, before the start of the actuation of the operating element, until the reaching of the intermediate value and thus the next compression step, in particular, a period of 2-5 s can be provided. If the compression force is reduced, i.e. on reducing the compression by one step, exactly the same procedure can be used. However, with a corresponding configuration of the operating element and the control facility, embodiments are conceivable in which the opposite takes place, that is, a temporary reduction below the base value to an intermediate value takes place in order then to increase it again, in particular abruptly, to the base value, in order here also to describe the reduction procedure haptically and intuitively.

Exemplary embodiments are also conceivable in which the variation starting from the base value to the intermediate value and back again which forms a first resistance value at each time point, forms only one actuation resistance component and the control facility for establishing the actuation resistance for the setting of which the actuator is activated, is configured as the sum of the first resistance value and of a second resistance value. Particularly advantageously, the second resistance value can be established from the item of compression information such that the second resistance value increases on an increase of the compression force and is reduced during a reduction of the compression force. In this way, therefore, a second resistance value rising and falling with the compression force is overlaid upon the variation referred to above in order to indicate the stepped increase and reduction. The second resistance value can be interpolated via the variation as far as the second resistance value achieved with the next compression step, for example, increased linearly to it, or it is conceivable, however, that the difference between the base value and the intermediate value is greater, in particular, at least twice as great as the difference between the second resistance values that arises during a compression step. Thus, after achievement of the intermediate value, the variation falls to the new sum of the base value and the second resistance value achieved by increasing or reducing the compression force by the step.

This variant offers the advantage that the actuation of the operating element remains the same for the operating person regardless of the number of compression steps, which means a lower loading and a smaller effort for the operating person, while at the same time, he is given an impression, by way of the awareness of completed increases/reductions, of the compression force acting upon the breast of the examination person.

In a second variant, the actuation of the operating element to increase the compression force by the next step, on reaching the next step of the compression force, the control facility can transfer control signals to the actuator regarding an output of the vibration and/or of the pulse to indicate the reaching of the next step, wherein the actuation resistance after reaching the next step of the compression force is preferably greater than before the reaching of this step. In a preferred combination with the configuration that the actuation resistance increases with an increase of the compression force and reduces with a reduction of the compression force, the actuation resistance increases continuously for each compression step so that, after reaching the next compression step, the actuation resistance is greater than before reaching this compression step and thus indicates the compression force that is acting. If the compression force is increased, for example, in each step by 2 N and if, according to the above example, the actuation resistance is initially 10 N (corresponding to 1 kg), the actuation resistance can increase until the next compression step is reached, for example, by 20 N, in corresponding steps. On reaching the next compression step, this can be indicated to the operating person by way of the output of a vibration and/or a pulse via the operating element. After reaching this compression step, the actuation resistance can be maintained, i.e. in this case, on a further increase of the compression force by a second compression step, the actuation resistance would start at 30 N (corresponding to 3 kg). On a further increase, the increment in the actuation resistance of 20 N is added to the already existing actuation resistance, so that overall, after achieving the second compression step, a total actuation resistance of 50 N (corresponding to 5 kg) acts.

Herein also, the increase to the next step can take place after a particular actuation travel and/or a particular actuation time. For example, from the start of the actuation of the operating element, until the reaching of the next compression step, a period of 2-5 s can be provided. If, expressed in general terms, the actuation is not carried out up to the next compression step which is advantageously indicated by the vibration or the pulse, an actuation resistance increase that takes place can be reversed again up to the time point of the actuation end. In particular, the vibration or the pulse thus indicates when an increase and/or a reduction by a step has actually been achieved and the actuation can thus, in particular, be ended if desired. On reducing the compression force, i.e. on reducing the compression by the next step, if the actuation resistance is to describe the current compression force, the opposite procedure to that described above can be used so that on each reduction of the compression force by a step, the actuating resistance also falls in dependence thereon.

With the provision of the actuating force dependent upon the compression force, this variant offers the advantage of a yet more precise and more intuitive operation since, due to the increasing actuation resistances with increasing compression force and thus number of compression steps, a still better relative impression can be imparted to the operating person regarding the compression force acting upon the breast of the examination person.

It is also conceivable that, via a user input, a selection can be made between the two variants. It can be suitable therein that at the start of the fine setting, one of the two variants is selected by the user.

In a further development of one or more example embodiments of the present invention, the operating element can be a foot pedal. However, it is optionally also possible that the operating element is a hand-operated regulator, for example, a twist knob or a press button or a lever. The use of a foot pedal as the operating element offers the advantage that the operating person has both hands free and so can possibly take care of the examination person and, for example, reassure her.

According to one or more example embodiments of the present invention, the foot pedal can have at least one holding device (or holding means) for holding the foot pedal after setting the compression force of the compression facility in a current actuation position. The holding device can be, for example, a clamping device (or clamping means), a locking device (or locking means) with different locking settings or a braking device (or braking means).

In the starting position, i.e. on non-actuation of the foot pedal, the surface serving for the placement of the foot can have an incline relative to the floor. Accordingly, in the starting position, the foot pedal can have an angle of 10° to 50° to the horizontal. After actuation of the foot pedal, i.e. after increasing the compression force, the foot pedal is no longer in the starting position, but rather, depending upon the compression force, has an angle to the horizontal reduced by a particular angle. If the foot pedal has an angle of 45° to the horizontal in a starting position, i.e. on non-actuation, then after actuation of the foot pedal and on reaching the next compression step, the angle can be reduced, for example, by 10°, so that the angle is now only 35°. Via the holding device, the foot pedal can be held at this angle and/or in this actuation setting, wherein on a further actuation of the foot pedal and thus a further increase in the compression force, the angle is further reduced until the foot pedal is fully depressed and the angle to the horizontal is 0° (or possibly less). Therein, the angle correlates with the compression force to be set. This means that, for example, for an increase of the compression force by 10 N, the angle can be reduced by a greater amount than, for example, for an increase in the compression force by 5 N. If the compression force is reduced, the procedure can take place in the reverse manner. I.e. in this case, the angle can be increased as far as the starting position of 45°.

The holding device offers the advantage that, with a corresponding operating concept, the operating person does not have to remain standing on the foot pedal or actively press the foot pedal in order to maintain the compression facility in a particular position, at which a specific compression force acts upon the breast of the examination person. Thus, the operating person can be freed, while the compression force on the breast is maintained and thereby a high quality imaging can be enabled.

As a further option, it can also be considered that, on ending the actuation, for example, the setting of a next compression step, the foot pedal returns to its starting position while the compression force of the compression facility on the breast of the examination person is simultaneously maintained. In this case, in order to increase the compression force, the operating person can press the foot pedal down, in particular, fully, whereby the compression force increases by the next step and subsequently the foot pedal can return into its starting position, for example, at an angle of 45°. Subsequently, by way of a further, in particular, complete pressing down of the foot pedal, in particular, against a actuation resistance increased correspondingly as haptic feedback, the compression force can be increased by the next compression step.

Preferably, the foot pedal can have a hooking-in device (or hooking-in device) for a foot placed thereon such that the foot pedal can be actuated by the foot in two opposing actuating directions of which one is associated with the increasing and one with the reducing of the compression force. In order to ensure that the foot does not slip off, nor is lifted off, the foot pedal during the actuating procedure, a hooking-in device can be provided which can be, in particular, a cap arranged on the placement area of the foot pedal. This cap is preferably closed on five sides and has only one, in a plan view onto the foot pedal, rear opening for receiving at least the toe area of the foot. Also conceivable is a bracket extending over the placement area. Optionally thereto, the placement area of the foot pedal can also be coated with a material that creates a high level of friction. The placement area can therein be coated, for example, with a rubber material or a material having particles to increase the frictional value or a material having a profile.

Suitably, in addition to at least one actuating direction, the foot pedal can have at least one additional actuating degree of freedom wherein the control facility is configured, on actuation of the foot pedal in the actuating direction, to activate the compression facility to the coarse setting of the compression force and, on actuation of the foot pedal in the additional actuating degree of freedom, to activate it to the fine setting of the compression force, in particular, in steps. The at least one actuating direction can herein comprise the pressing down of the placement area of the foot pedal for the foot and/or the lifting up of the placement area of the foot pedal. By way of the provision of a further actuating degree of freedom, in effect, the fine setting taking place, in particular, by steps, that is, in particular, the incremental adaptation of the compression can, particularly advantageously also be physically integrated into the foot pedal, so that the fine setting does not have to be coupled, as in the case described above, to a change of the operating mode, but rather can be used, in particular, at any time. The operating person can control the compression in the preferred case that both actuating directions are used, also completely via a single operating element, specifically the foot pedal, with respect both to the coarse setting and also the fine setting. Thus, the operating person can focus his hands on the patient.

The additional actuating degrees of freedom can generally be selected, particularly advantageously, from the group comprising:
movement of the foot to touch an actuating portion on the right and/or left side of the placement area,
lateral deflectability of the foot pedal, in particular, by way of rotation or pivoting about a rotation axis extending perpendicularly to the placement area, wherein the intensity of the deflection preferably increases toward the toe-side end of the placement area,
forward and/or backward movement capability of the placement area along its longitudinal axis,
tilting capability of the placement area about its longitudinal axis.

During the use of these actuating degrees of freedom that provide, in particular, symmetrical actuating capabilities, it is, in principle, conceivable that if the foot pedal serves either only to increase or only to reduce the compression force, in a particularly advantageous configuration of one or more example embodiments of the present invention, they are used when the foot pedal can be actuated both for increasing and also for reducing the compression force. For this purpose, for the coarse setting in the control facility, the actuating directions can be associated respectively with the increasing and the reducing of the compression force. For the fine setting, for example, during increasing of the compression force, the foot pedal and/or specifically the placement area for the foot can be rotated and/or tilted in a first direction of the additional actuating degree of freedom, and/or the placement area for the foot can be displaced forwardly. Optionally, just the foot can also be displaced in a first direction on the placement area. Conversely thereto, for a reduction of the compression force, the placement area for the foot can be rotated and/or tilted in the second direction of the additional actuating degree of freedom, and/or the placement area for the foot can be displaced rearwardly. Optionally, for this, just the foot can also be displaced in the second direction on the placement area. The first direction can be, for example, right and the second, left. It is naturally also conceivable that the compression force is increased by a left-sided rotation and/or inclination and/or movement and/or a displacement rearwardly and the compression force is reduced by a right-sided rotation and/or inclination and/or movement and/or displacement forwardly.

It should be noted at this point that at least one actuator can be associated with both the at least one movement direction and also the at least one additional actuating degree of freedom in order to provide haptic feedback on the compression force and/or its change for all the actuating types. Therein, a common actuator can also be used both for the coarse setting and the fine setting, in particular, associated with the placement area. Such a common actuator can serve, for example, for vibration of the placement area.

If two foot pedals are used for the setting of the compression force so that a first foot pedal serves for increasing the compression force and a second foot pedal for reducing the compression force, the fine setting can be carried out, in particular, by way of a double actuation (“double tap”) of the respective foot pedal. Thus, for example, on a change from the first operating mode into the second operating mode, for further increasing the compression force, the first foot pedal can be actuated twice in quick succession in order to undertake a further setting.

One or more example embodiments of the present invention further relate to the operation of a mammography apparatus comprising a control facility, an object support for placement of a breast of an examination person, a compression facility for compressing the breast arranged on the object support, at least one operating element for increasing and/or reducing a compression force acting upon the breast of the examination person and an acquisition facility for acquiring an item of compression information, which defines the currently acting compression force, having the subsequent step that, dependent upon the item of compression information, an actuator of the operating element is activated via which a haptic information item is output to provide a haptic feedback.

All the method-specific and apparatus-specific features that have been disclosed for the mammography apparatus, according to example embodiments of the present invention, naturally apply also for the mammography apparatus used in the method. This applies equally for all the alternative embodiments described and for all the combinations thereof, provided they are not incompatible.

### BRIEF DESCRIPTION OF THE DRAWINGS

Further advantages and details of the present invention are disclosed in the exemplary embodiments described below and by reference to the drawings. In the drawings:

FIG. 1 shows a schematic illustration of the principle of a first exemplary embodiment of a mammography apparatus according to the present invention,

FIG. 2 shows a representation of a change of an actuation resistance in dependence upon time in the context of a first variant,

FIG. 3 shows a representation of a change of an actuation resistance in dependence upon time in the context of a second variant,

FIG. 4 shows a schematic side view of a foot pedal in the first exemplary embodiment,

FIG. 5 shows a schematic view of a foot pedal of a second exemplary embodiment of the mammography apparatus according to the present invention in a first possible configuration,

FIG. 6 shows a schematic view of a foot pedal of the second exemplary embodiment in a second possible configuration,

FIG. 7a shows a schematic view of a foot pedal of the second exemplary embodiment in a third possible configuration,

FIG. 7b shows a schematic side view of the foot pedal of FIG. 7a,

FIG. 8 shows a schematic front view of a foot pedal of the second exemplary embodiment in a fourth possible configuration, and

FIG. 9 shows a schematic flow diagram of a method according to the present invention for operating the mammography apparatus.

### DETAILED DESCRIPTION

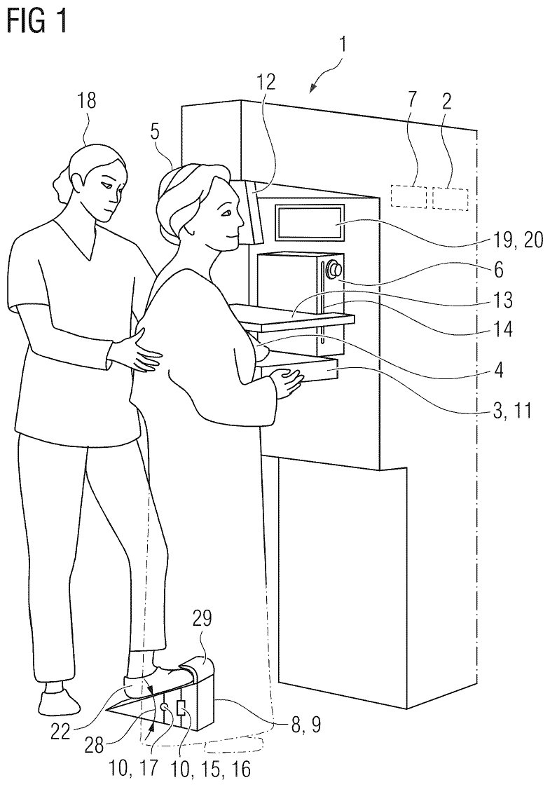

FIG. 1 shows a mammography apparatus 1 according to one or more example embodiments of the present invention, comprising a control facility 2, an object support 3 for placement of a breast 4 of an examination person 5, a compression facility 6 for compressing the breast 4 arranged on the object support, at least one operating element 8 that is configured as a foot pedal 9, an acquisition facility 7 for acquiring an item of compression information, which defines the currently acting compression force, and an actuator 10 arranged in the foot pedal 9. In the present case, the object support 3 also comprises the X-ray detector 11 of a recording arrangement of the mammography facility 1 and/or is formed thereby. The recording arrangement further also comprises an X-ray radiator 12. The compression facility 6 for compressing a breast from which an image dataset is to be recorded by the recording arrangement comprises, in particular, a compression plate 13 (often called a paddle) which can be moved in a linear guideway 14 via a movement device (or movement means) perpendicularly to the object support 3 such that different spacings therefrom result. In this regard, the acquisition facility 7 can suitably comprise a compression sensor which can be arranged, for example, on the object support 3 or on the compression plate 13. The currently acting compression force can, however, also be determined by another device, mechanism and/or means, for example, using operating variables of the compression facility 6. The control facility 2 can activate the movement device as a reaction to operating input on the foot pedal 9 in order to increase or reduce the compression force according to the wish of an operating person 18.

The control facility 2 is configured such that it activates the actuator 10 dependent upon the item of compression information for generating a haptic feedback. The actuator 10 has both a setting element 15, for example, an actuating cylinder 16 and also a motor arrangement 17. The mammography apparatus 1 is operated, in particular, by the operating person 18.

At the start of the mammographic screening, the breast 4 of the examination person 5 is placed on the object support 3 and the operating person 18 starts the screening in a first operating mode. The first operating mode serves for the coarse setting of the compression force. Therein, the compression force can be set steplessly by way of the actuation of the foot pedal 9 by the operating person 18, wherein dependent upon a change in the compression force (described by the item of compression information), a changed actuation resistance is output via the actuator 10 and, in particular, via the setting element 15 via the foot pedal 9, as a haptic feedback signal. Accordingly, the actuation resistance rises when the compression force is increased and falls when the compression force is reduced. Meanwhile, the operating person 18 has sight of the examination person 5 to ascertain whether, for example, the examination person 5 feels unwell due to pain. Dependent upon the properties of the breast and upon image recording parameters, for example, the dimensionality of the image dataset to be recorded, a different compression force is required in order to enable a qualitatively high-value imaging for diagnostic evaluation. In the present exemplary embodiment, a compression range that is specific to the current mammography examination is used. The specific compression range, which can also be designated a critical compression range, is suitably selected so that its lower limit specifies that a sufficient fixation is provided for the imaging and should still be tolerable for the examination person. For example, such a specific compression range can be at 50 N, which corresponds approximately to a weight force from a mass of 5 kg.

Based upon techniques such as OpComp, the achievement of the specific compression range can be recognized on fulfillment of a notification condition which indicates that a change in the breast thickness with an increase in the compression force, in particular, a corresponding gradient, is below a limit value. Other possibilities for defining the compression range clearly in which a fine setting is to be undertaken are also naturally conceivable and corresponding notification conditions can be formulated. In general, the compression range is an optimum range for imaging which naturally can also be dependent upon image recording parameters, for example, the dimensionality. In the exemplary case described above, the limit value can be selected differently, for example, for 2D imaging and 3D imaging.

On entry into the specific compression range, that is, when the notification condition is fulfilled, the motor arrangement 17 is actuated by the control facility 2 for the output of a vibration as a haptic feedback, whereby it is signaled to the operating person 18 that he has now reached the specific compression range and that a coarse setting in the first operating mode is complete. In the present exemplary embodiment, the compression force can, however, be further increased to improve the image quality. Therefore, on fulfillment of the notification condition, immediately or after a predetermined waiting time, for example, 5 seconds, in the compression range set by way of the coarse setting via the control facility 2, a changeover takes place from the first operating mode into a second operating mode with which, via the foot pedal 9, a fine setting in steps can be undertaken.

If the compression force is to be increased, for example, in steps of 2 N, this can be predetermined or it is input by the operating person 18 into a specifying facility 19, here on a touch display 20 which is arranged on the mammography apparatus 1. Optionally, another step size can also be defined in the region, in particular, of 0.1 N to 2 N. On corresponding actuation of the foot pedal 8, the compression force is subsequently increased by 2 N, whereby herein via the actuating cylinder 10, just as in the first operating mode, an actuation resistance is output as a haptic feedback signal via the foot pedal 8. During the fine setting, therefore an actuation resistance is output to the operating person 18 until the next compression step is reached, in this case 2 N, which actuation resistance is intended to impart to the operating person 18 a relative impression of the compression force acting upon the breast 4 of the examination person 5. For this purpose, in particular, the actuation resistance itself can reduce the compression force currently acting, or, in particular, as the height of a threshold to be overcome, reduce the step height, that is, the compression force change. For example, the actuation resistance can be in an actuation resistance range from 1 N to 400 N.

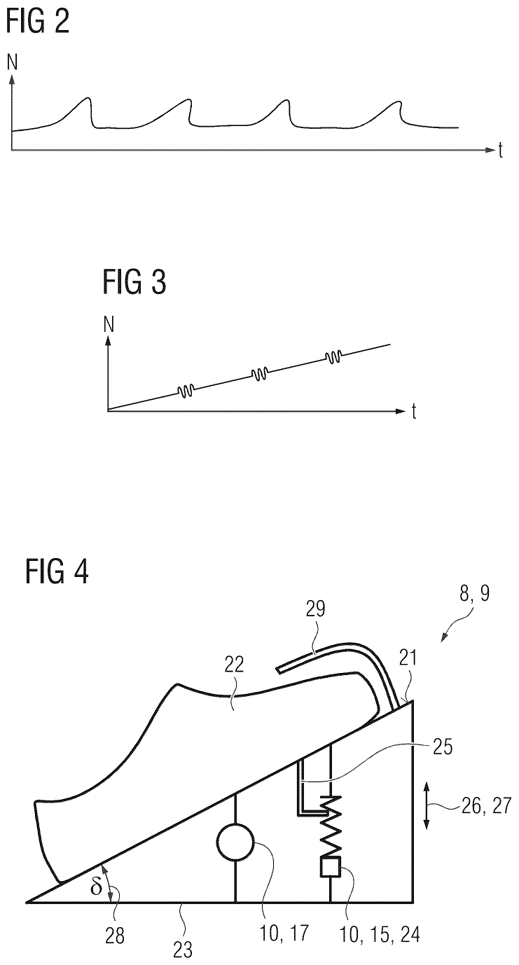

FIG. 2 shows, for a first variant, a change in the actuation resistance over time. Herein, the actuation resistance on non-actuation of the operating element 8 has a base value which is, in particular, 10 N. If the operating person 18 now actuates the foot pedal 9 in order to increase the compression force, the control facility 2 increases the actuation resistance by activating the actuating cylinder 16, starting from the base value, in such a way that the operating person 18 must overcome a type of threshold in order to reach the next compression step. An intermediate value of the actuation resistance then effectively corresponds to the height of this threshold. If the intermediate value is, for example, 100 N, the actuation resistance increases, for example, in steps, but preferably continuously until the intermediate value is reached at an actuation resistance of 100 N. At this time point, the control facility 2 controls the compression facility 6 to increase the compression force by one step. Once the next compression step is reached in this way, the actuation resistance falls off abruptly so that it corresponds again to the actuation resistance in the starting position, i.e. the base value of, in particular, 10 N.

FIG. 3 shows, for a second variant, a change in the actuation resistance over time. Here, the actuation resistance increases continuously for each compression step. At the start, the actuation resistance is, for example, 10 N. If the operating person 18 actuates the foot pedal 9 in order to increase the compression force by 2 N, the actuation resistance increases until the next compression step is reached, for example, by 20 N (specifically, therefore for example, ten times the step size of 2 N). On reaching the actuation resistance of 30 N, the control facility 2 activates the compression facility 6 to increase the compression force by one compression step. Via the motor arrangement 17, a vibration is output, so that it is signaled to the operating person 18 that he has now reached the next compression step. If, however, it is necessary for the compression force again to be increased by 2 N, since the image quality can, for example, be enhanced and the examination person 5 still tolerates the compression, the actuation resistance at the start of the increase of the compression force by the next, here the second, step is already 30 N. This is again increased by 20 N so that after reaching an actuation resistance of 50 N, the control facility 2 again increases by one compression step and activates the motor arrangement 17 to output a vibration. In this case, the actuation resistances of the individual compression steps are thus summed so that after each increase in the compression force, there is a higher actuation resistance for the operating person 18.

FIG. 4 shows the foot pedal 9 with a placement area 21 for a foot 22, and a body 23. Via the foot pedal 9, the increase and/or reduction of the compression force acting by way of the compression facility 6 is set. The foot pedal 9 has a spring arrangement 24 as the setting element 15 which is configured for setting the actuation resistance, and the motor arrangement 17 which is configured for the output of the vibration. In order to hold the foot pedal after setting the compression force of the compression facility 6 in a current actuation setting, the foot pedal 9 has a holding device 25 (also referred to as a retaining device 25), which in the present case is a detent device, for example, a hook which holds the placement area 21 in the current actuation setting.

In the starting position, i.e. on non-actuation, the foot pedal 9 has an angle 28 of, in particular, 45° which is formed between the placement area 21 and a part of the body 23 of the foot pedal 9 which is oriented, in particular, horizontally. On actuating the foot pedal 9, for example, in order to increase the compression force, with every increase of the compression step, the angle 28 is reduced by a particular number of degrees until the placement area 21 of the foot pedal 9 lies completely flat on the body 23 and the angle 28 is therefore 0°. In this actuation setting, the highest compression is also achieved and a further increase of the compression force is not possible. On reducing the compression force, inversely thereto, the angle 28 is increased by a particular number of degrees until the placement area 21 is again in the starting position and is spaced by, in particular, 45° from the body 23. The actuating directions of the foot pedal 9 for increasing and/or reducing the compression force are indicated by arrows 26, 27.

The foot pedal 9 also has a hooking-in device 29. In the present exemplary embodiment, the hooking-in device 29 is a cap for receiving at least the toe region of the foot 22, wherein the cap is arranged on the placement area 21 of the foot pedal 9. In a side view of the foot pedal 9 as shown in FIG. 2, the cap is arranged such that in a starting position of the foot pedal 9, it is situated at the end of the placement area 21 that is situated in a raised position. In addition, the foot pedal 9, in particular, the placement area 21 can also be coated with a material to increase the friction in order to prevent slipping of the foot 22 off the foot pedal 9.

In the first exemplary embodiment described making reference to FIGS. 1 to 4, the actuating directions are used both for fine setting and also for coarse setting, for which two operating modes are used. In the second exemplary embodiment described below, a foot pedal 9 is used which also permits fine and coarse setting by way of the provision of additional actuating degrees of freedom at the same time.

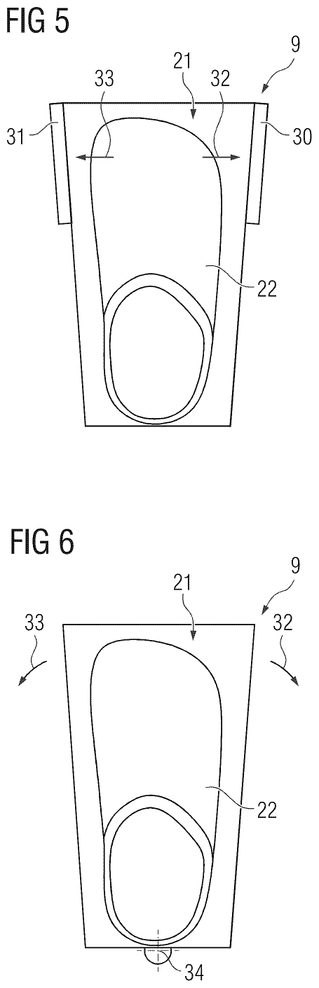

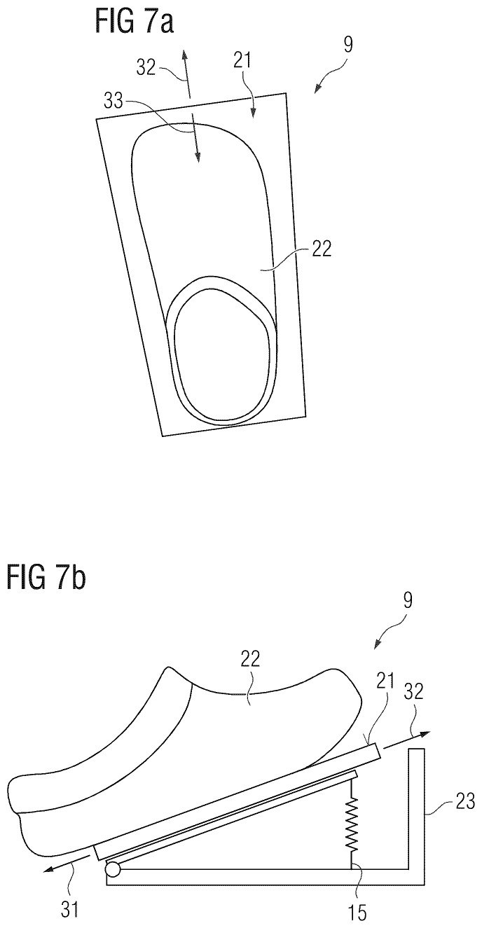

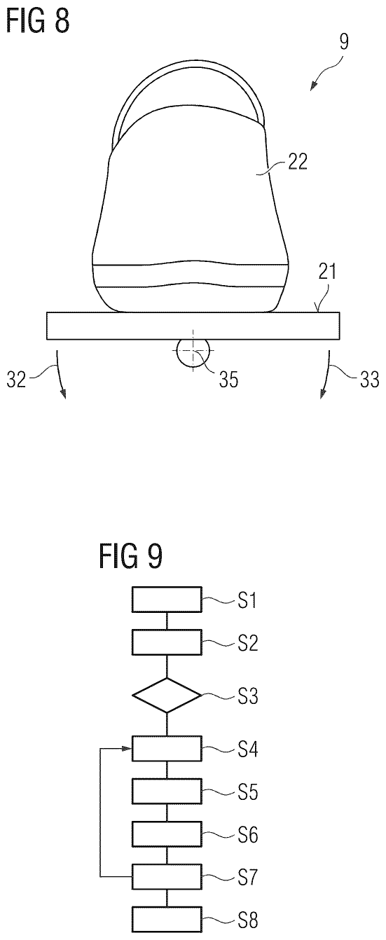

FIGS. 5 to 8 show possible configurations of the additional actuating degrees of freedom of the foot pedal 9 which can be implemented in the context of the second exemplary embodiment in order to set the compression force, independently of an automatic change of the operating modes on fulfillment of a notification condition, in the context of the fine setting. As shown in FIG. 5, by touching an actuating portion 30 on the right side of the placement area 21 of the foot pedal 9, the compression force is increased and by touching the actuating portion 31 on the left side, the compression force is reduced. The actuating degree of freedom for increasing the compression force during the fine setting is represented by an arrow 32; the actuating degree of freedom for reducing the compression force is represented by an arrow 33. Optionally thereto, the compression force can also be increased and/or reduced by way of a right-sided and/or left-sided rotation about a rotation axis 34 (FIG. 6) extending perpendicularly to the placement area 21, a displacement of the placement area 21 forwardly and/or rearwardly (FIGS. 7a and 7b) and an inclination of the placement area 21 about a longitudinal axis 35 (FIG. 8).

FIG. 9 shows a schematic flow diagram of a method for operating a mammography apparatus 1. Therein, in a first step S1, the breast 4 of the examination person 5 is arranged on the object support 3.

Subsequently, in a second step S2, the operating person 18 can make a coarse setting of the compression force in a first operating mode by operating the foot pedal 9.

In a step S3, the fulfillment of the notification condition is checked, that is, it is ascertained whether the specific compression range has been entered by way of the coarse setting. If this is fulfilled, the vibration is output as haptic feedback and the first operating mode is ended.

If the compression force established via the coarse setting is suitable for the image recording, for example, for assessing a potential mammary carcinoma, and simultaneously the pain perception of the examination person 5 is already at the limit of tolerability, the method is ended in this third step S3. If, however, after the coarse setting, a further compression of the breast 4 is possible since the examination person 5 tolerates a greater compression, the operating person 18 can further increase the compression force at his discretion in order to achieve an improvement in the image quality. After an optional waiting time of, for example, 5 seconds in which the foot pedal 9 cannot be actuated, the control facility 2 for the foot pedal 9 changes out of the first operating mode into the second operating mode in which a fine setting can be undertaken in steps.

In the fourth step S4, the operating person 18 can select the step size of the compression force that needs to be set further, via the specifying facility 19. This selection can also take place as soon as the start of the method and/or beforehand.

In the fifth step S5, the operating person 18 can actuate the foot pedal 9 during the fine setting in order to increase or reduce the compression force further. Therein, when actuating the foot pedal 9, according to the first or second variant, in a step S6, a haptic feedback is output via the foot pedal 8 so that the operating person 18 receives a relative impression regarding the compression force acting upon the breast 4 of the examination person 5. The fine setting is continued until a tolerable compression force with the best possible image quality is found.

Subsequently, in a seventh step S7, an image recording of the mammography examination can take place.

Subsequently, in an eighth step S8, by actuating the foot pedal 9 or another operating element for releasing the breast 4, the compression force is reduced until no compression force acts upon the breast 4 of the examination person 5 and thus the mammography examination is concluded.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic@, Lua, and Python@.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A mammography apparatus, comprising:
a control device;
an object support for placement of a breast of a patient;
a compression device to compress the breast arranged on the object support;
at least one operating element to at least one of increase or decrease a compression force acting on the breast; and
an acquisition device to acquire an item of compression information describing the compression force currently acting on the breast, wherein
the at least one operating element includes an actuator to output haptic information, and
the control device is configured to activate the actuator based on the item of compression information to generate haptic feedback.

2. The mammography apparatus as claimed in claim 1, wherein
the haptic feedback includes at least one of a vibration output, a pulse output, or a change in an actuation resistance of the at least one operating element, and
the actuator includes at least one of (i) a setting element to set the actuation resistance or (ii) a motor arrangement to provide at least one of the vibration output or the pulse output.

3. The mammography apparatus as claimed in claim 2, wherein the control device is configured to establish the actuation resistance via the actuator based on the compression force, such that the actuation resistance increases as the compression force is increased and the actuation resistance decreases as the compression force is decreased.

4. The mammography apparatus as claimed in claim 2, wherein the control device is configured to activate the actuator to output at least one of the vibration output or the pulse output when a notification condition is fulfilled, the notification condition indicating that the compression force is within a compression range.

5. The mammography apparatus as claimed in claim 4, wherein the control device is configured, on fulfillment of the notification condition, to change an operating mode of the at least one operating element from a first operating mode to a second operating mode provided the compression force remains in the compression range.

6. The mammography apparatus as claimed in claim 5, wherein the first operating mode is provided for coarse setting and the second operating mode is provided for fine setting.

7. The mammography apparatus as claimed in claim 2, wherein, in at least one stepped operating mode of the at least one operating element, the control device is configured to at least one of increase or decrease the compression force in steps upon actuation of the at least one operating element.

8. The mammography apparatus as claimed in claim 7, wherein
the actuation resistance output by the actuator on non-actuation of the at least one operating element has a base value at least in the at least one stepped operating mode, and
the control device is configured to
reduce the actuation resistance on operation of the at least one operating element to increase the compression force by a next step to an intermediate value, and
reduce the actuation resistance to the base value upon reaching the next step.

9. The mammography apparatus as claimed in claim 7, wherein, upon actuation of the at least one operating element to increase the compression force by a next step, the control device is configured to transfer control signals to the actuator upon reaching the next step, the control signals regarding at least one of the vibration output or the pulse output to indicate reaching the next step.

10. The mammography apparatus as claimed in claim 1, wherein the at least one operating element is a foot pedal.

11. The mammography apparatus as claimed in claim 10, wherein the foot pedal has at least one holding device to hold the foot pedal after setting the compression force of the compression device at a current actuation setting.

12. The mammography apparatus as claimed in claim 10, wherein the foot pedal has a hooking-in device for a foot placed thereon such that the foot pedal is configured to be actuated by the foot in two opposing actuating directions, a first of the two opposing actuating directions being associated with increasing of the compression force and a second of the two opposing actuating directions being associated with decreasing of the compression force.

13. The mammography apparatus as claimed in claim 10, wherein
the foot pedal has at least one additional actuating degree of freedom in addition to at least one actuating direction, and
the control device is configured to,
activate, on actuation of the foot pedal in the at least one actuating direction, the compression device to a coarse setting of the compression force, and
activate, on actuation of the foot pedal in the at least one additional actuating degree of freedom, the compression device to a fine setting of the compression force.

14. A method for operating a mammography apparatus including a control device, an object support for placement of a breast of a patient, a compression device to compress the breast arranged on the object support, at least one operating element to at least one of increase or decrease a compression force acting upon the breast, and an acquisition device to acquire an item of compression information describing the compression force currently acting on the breast, the method comprising:
activating, based on the item of compression information, an actuator of the at least one operating element to output an item of haptic information to provide haptic feedback.

15. The mammography apparatus as claimed in claim 5, wherein the first operating mode of the at least one operating element is for continuous setting of the compression force.

16. The mammography apparatus as claimed in claim 5, wherein the second operating mode is a stepped operating mode.

17. The mammography apparatus as claimed in claim 3, wherein the control device is configured to activate the actuator to output at least one of the vibration output or the pulse output when a notification condition is fulfilled, the notification condition indicating that the compression force is within a compression range.

18. The mammography apparatus as claimed in claim 3, wherein, in at least one stepped operating mode of the at least one operating element, the control device is configured to at least one of increase or decrease the compression force in steps upon actuation of the at least one operating element.

19. The mammography apparatus as claimed in claim 11, wherein
the foot pedal has at least one additional actuating degree of freedom in addition to at least one actuating direction, and
the control device is configured to,
activate, on actuation of the foot pedal in the at least one actuating direction, the compression device to a coarse setting of the compression force, and
activate, on actuation of the foot pedal in the at least one additional actuating degree of freedom, the compression device to a fine setting of the compression force.

20. The mammography apparatus as claimed in claim 12, wherein
the foot pedal has at least one additional actuating degree of freedom, and
the control device is configured to,
activate, on actuation of the foot pedal in at least one of the two opposing actuating directions, the compression device to a coarse setting of the compression force, and
activate, on actuation of the foot pedal in the at least one additional actuating degree of freedom, the compression device to a fine setting of the compression force.
